(19) United States
(12) Patent Application Publication
(10) Pub. No.: US 2013/0155215 A1
(43) Pub. Date: Jun. 20, 2013

(54) Scanning Endoscope Device

(71) Applicant: Olympus Medical Systems Corp., Tokyo (JP)

(72) Inventors: Tomoko Shimada, Tokyo (JP); Masahiro Yoshino, Tokyo (JP); Nobuyuki Doguchi, Tokyo (JP); Shunji Takei, Tokyo (JP)

(73) Assignee: OLYMPUS MEDICAL SYSTEMS CORP., Tokyo (JP)

(21) Appl. No.: 13/633,504

(22) Filed: Oct. 2, 2012

Related U.S. Application Data
(63) Continuation of application No. PCT/JP2012/055231, filed on Mar. 1, 2012.

(30) Foreign Application Priority Data
Mar. 31, 2011 (JP) 2011-080635

(51) Int. Cl. A61B 1/06

(52) U.S. Cl. CPC A61B 1/0661
USPC 348/68

(57) ABSTRACT

Scanning endoscope device including a light source unit that repeatedly emits a plurality of illumination beams in different wavelength bands in a certain order with a predetermined repetition period; a light guiding section that has an emission surface; a driving section that oscillates the emission surface in two axial directions in a reciprocating manner to two-dimensionally scan the illumination beams; a controller that controls the source unit and/or the driving section so that an oscillation period and a oscillation amplitude of the emission surface are proportional to the period of the illumination beams, and that controls the driving section so that the oscillation period periodically and gradually changes and the oscillation period is proportional to the oscillation amplitude; a light detecting section that detects return beams; and an image generating section that generates images of the return beams in synchronization with the predetermined period of the source.

### CROSS-REFERENCE TO RELATED APPLICATIONS

This is a continuation of International Application PCT/JP2012/055231, with an international filing date of Mar. 1, 2012, which is hereby incorporated by reference herein in its entirety. This application claims the benefit of Japanese Patent Application No. 2011-080635, the content of which is incorporated herein by reference.

### TECHNICAL FIELD

The present invention relates to scanning endoscope devices.

### BACKGROUND ART

With regard to a scanning endoscope device in the related art that acquires a two-dimensional image by scanning illumination light along a spiral trajectory, a scanning endoscope device that detects the illumination light with a period that is inversely proportional to the distance from the center of the scan trajectory is known (for example, see Patent Literature 1). Such a scanning endoscope device solves the problem of the irradiation density of the illumination light radiated onto a subject becoming lower from the center of the scan trajectory toward the outer side thereof, thereby making the irradiation density of the illumination light uniform within a generated image.

Furthermore, in Patent Literature 1, white light having a mixture of light beams in red, green, and blue wavelength bands is radiated onto the subject, and the reflected light of the white light is separated into light components in the red, green, and blue wavelength bands. The separated light components are detected by multiple detectors. Based on the signal intensities corresponding to the quantities of light received by the detectors, R, G, and B single-color images are generated. By superposing these R, G, and B single-color images, a color image can be generated.

### CITATION LIST

### Patent Literature

### {PTL 1}

### Japanese Unexamined Patent Application, Publication No. 2010-142482

### SUMMARY OF INVENTION

### Technical Problem

When images of multiple illumination light beams having different wavelengths are to be acquired by repeatedly irradiating the subject with the illumination light beams in a certain order, the irradiation density can be made uniform by detecting the illumination light beams with a period that is inversely proportional to the distance from the center, as in the device according to Patent Literature 1. Because the device according to Patent Literature 1 is not made in view of the repetition period of the wavelengths, illumination light beams that change with time cannot be detected at an appropriate timing. Therefore, different colors are displayed at misaligned positions (i.e., color misregistration occurs) in the color image formed of superposed single-color images.

### Solution to Problem

The present invention provides a scanning endoscope device including an insertion section that is inserted into a subject; a light source unit that repeatedly emits a plurality of illumination light beams in different wavelength bands in a certain order with a predetermined repetition period; a light guiding section that is provided within the insertion section and has an emission surface that causes the illumination light beams from the light source unit to be emitted from an end of the insertion section; a driving section that oscillates the emission surface in two axial directions, which intersect a longitudinal direction of the insertion section, in a reciprocating manner so as to two-dimensionally scan the illumination light beams; a controller that controls at least one of the light source unit and the driving section so that an oscillation period of the emission surface and a oscillation amplitude of the emission surface are proportional to the predetermined repetition period of the illumination light beams, and that controls the driving section so that the oscillation period of the emission surface gradually changes and the oscillation period is proportional to the oscillation amplitude; a light detecting section that detects return light beams from the subject; and an image generating section that generates images of the return light beams in synchronization with the predetermined repetition period of the light source.

### BRIEF DESCRIPTION OF DRAWINGS

FIG. 1 illustrates the overall configuration of a scanning endoscope device according to an embodiment of the present invention.

FIG. 2 is an enlarged view of a distal end of an insertion section included in the scanning endoscope device in FIG. 1.

FIG. 3 illustrates a driving voltage applied to an actuator in the scanning endoscope device in FIG. 1.

FIG. 4 schematically illustrates a scan trajectory of illumination light beams and irradiation positions of the illumination light beams according to the scanning endoscope device in FIG. 1.

FIG. 5 illustrates a modification of the scanning endoscope device in FIG. 1.

FIG. 6 illustrates another modification of the scanning endoscope device in FIG. 1.

### DESCRIPTION OF EMBODIMENTS

A scanning endoscope device 1 according to an embodiment of the present invention will be described below with reference to the drawings.

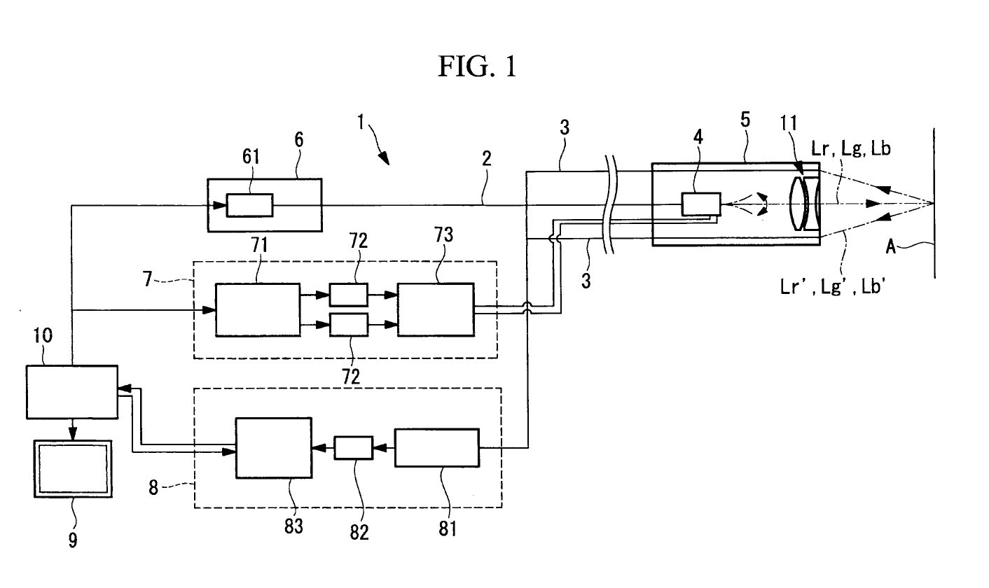

As shown in FIG. 1, the scanning endoscope device 1 according to this embodiment includes an illumination fiber (light guiding section) 2; an insertion section 5 having an actuator (driving section) 4 that vibrates distal ends of light-receiving fibers 3 and the illumination fiber 2; an illumination unit 6 that supplies illumination light beams Lr, Lg, and Lb to the illumination fiber 2; a driving unit 7 that drives the actuator 4; a detecting unit 8 that generates an image based on return light beams Lr′, Lg′, and Lb′ of the illumination light beams Lr, Lg, and Lb received by the light-receiving fibers 3, a control unit (controller) 10 that controls the operation of the illumination unit 6 and the driving unit 7 and outputs the image generated by the detecting unit 8 to a monitor 9.

The illumination fiber 2 and the light-receiving fibers 3 are longitudinally disposed within the insertion section 5. An illumination optical system 11 is provided at the distal end of the illumination fiber 2. The illumination fiber 2 guides the illumination light beams Lr, Lg, and Lb supplied from the illumination unit 6 at the base end thereof and emits the light beams from a distal-end surface (emission surface) thereof. The illumination light beams Lr, Lg, and Lb emitted from the distal-end surface are converged by the illumination optical system 11 before being radiated from the distal end of the insertion section 5 onto a tissue surface serving as an observation surface A within a living organism (subject).

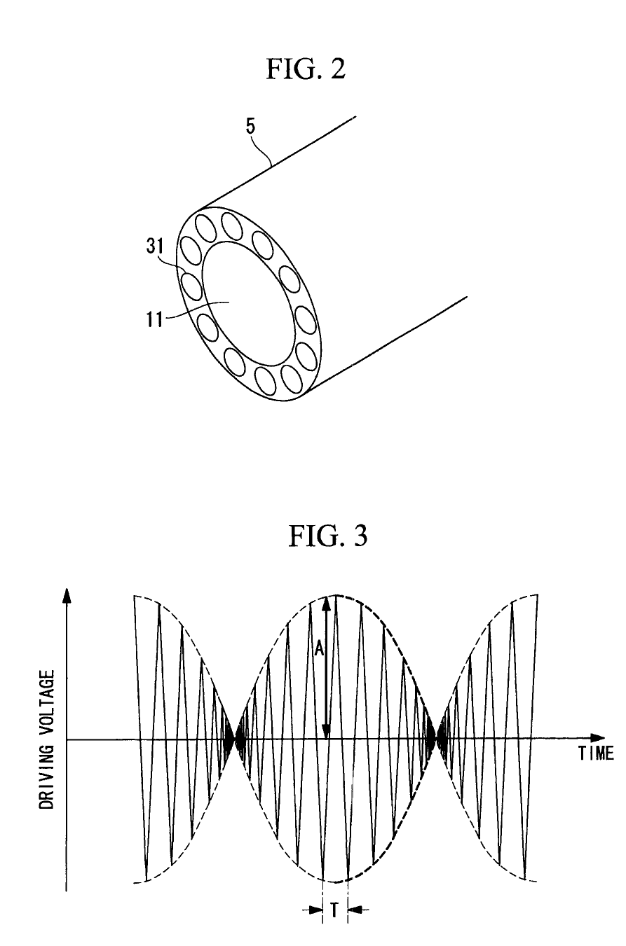

Each light-receiving fiber 3 has a light-receiving surface (light-receiving portion) 31 defined by a distal-end surface that collectively receives the return light beams Lr′, Lg′, and Lb′ from the observation surface A, and guides the light beams to the detecting unit 8. As shown in FIG. 2, there are multiple (12 in the shown example) light-receiving fibers 3. The light-receiving surfaces 31 are arranged on the distal-end surface of the insertion section 5 so as to surround the illumination optical system 11 in the circumferential direction thereof. Thus, the level of the return light beams Lr′, Lg′, and Lb′ received by the light-receiving fibers 3 is increased.

The actuator 4 is of, for example, an electromagnetic type or a piezoelectric type. The actuator 4 receives alternating voltages in the X direction and the Y direction as driving voltages (to be described later) from the driving unit 7. The actuator 4 vibrates the distal end of the illumination fiber 2 in two axial directions (i.e., the X direction and the Y direction), which intersect the longitudinal direction of the illumination fiber 2 and are perpendicular to each other, with frequencies and amplitudes according to the driving voltages. Thus, the distal-end surface of the illumination fiber 2 is oscillated in the two axial directions, whereby the illumination light beams Lr, Lg, and Lb emitted from the distal-end surface are two-dimensionally scanned over the observation surface A.

The illumination unit 6 includes a wavelength-swept light source 61 that emits illumination light beams while changing the wavelengths thereof. In accordance with a command from the control unit 10, the wavelength-swept light source 61 repeatedly emits, for example, the three illumination light beams Lr, Lg, and Lb in red, green, and blue wavelength bands in a certain order with a fixed repetition period at fixed time intervals. The illumination light beams Lr, Lg, and Lb emitted from the wavelength-swept light source 61 are input to the base end of the illumination fiber 2. The order in which the illumination light beams Lr, Lg, and Lb are emitted is not limited in particular; the illumination light beams may be emitted in the order: Lb, Lg, and Lr.

The driving unit 7 includes a signal generating section 71 that generates driving signals for driving the actuator 4 as digital signals, two D/A converting sections 72 that convert the driving signals generated by the signal generating section 71 into analog signals, and a signal amplifying section 73 that amplifies the outputs from the D/A converting sections 72.

In accordance with specifications (to be described later) designated by the control unit 10, the signal generating section 71 generates two driving signals for the X direction and the Y direction and inputs the two driving signals to the separate D/A converting sections 72.

The signal amplifying section 73 amplifies the analog signals generated by the D/A converting sections 72, that is, driving voltages, to levels suitable for driving the actuator 4, and outputs the analog signals to the actuator 4.

The detecting unit 8 includes a light detector 81 that detects the return light beams Lr′, Lg′, and Lb′ guided by the light-receiving fibers 3 and performs photoelectric conversion on the light beams, an A/D converting section 82 that converts photocurrents output from the light detector 81 into digital signals, and an image generating section 83 that generates two-dimensional images from the digital signals generated by the A/D converting section 82.

The magnitude of the photocurrents output from the light detector 81 to the A/D converting section 82 corresponds to the level of the detected return light beams Lr′, Lg′, and Lb′.

Based on information regarding the emission timing of the illumination light beams Lr, Lg, and Lb received from the control unit 10 and information regarding irradiation positions (to be described later), the image generating section 83 generates three two-dimensional images, namely, an R-image, a G-image, and a B-image, as original images from the digital signals received from the A/D converting section 82. Specifically, the image generating section 83 generates an R-image from the digital signal of the return light beam Lr′ detected by the light detector 81 when the red illumination light beam Lr is emitted from the illumination unit 6. Likewise, the image generating section 83 generates a G-image from the return light beam Lg′ and a B-image from the return light beam Lb′.

Then, the image generating section 83 displays the R-image, the G-image, and the B-image in red, green, and blue, respectively, and then superposes the R-image, the G-image, and the B-image so as to generate an RGB image (color image) for normal observation.

The image generating section 83 may generate a special-light image in addition to the RGB image. For example, by radiating the green illumination light beam Lg and the blue illumination light beam Lb, which are readily absorbable by hemoglobin in the blood, fine patterns of capillary vessels in the surface layer of a mucous membrane or fine patterns in a mucous membrane may be generated as a special-light observation image. Specifically, the blue wavelength band (ranging between 390 nm and 445 nm) may be used for observing the capillary vessels in the surface layer of a mucous membrane, and the green wavelength band (ranging between 530 nm and 550 nm) may be used for observing an image with emphasized contrast between thick blood vessels in deep areas and the capillary vessels in the surface layer of a mucous membrane. By generating a G′-image and a B′-image from return light beams of the illumination light beams Lg and Lb and superposing these images, a special-light observation image with emphasized contrast between the surface layer of a mucous membrane and the blood vessels in deep areas can be generated.

Furthermore, light beams with wavelengths other than the aforementioned wavelength absorbable by hemoglobin may be used as the illumination light beams for normal observation. For example, multiple illumination light beams, such as Lb1 (415 nm), Lb2 (450 nm), Lg1 (520 nm), Lg2 (540 nm), and Lr (635 nm), may be used. Accordingly, special-light observation can be performed simultaneously with the normal observation based on the RGB image. The RGB image and the special-light image may be displayed side-by-side or in a superposed fashion on the monitor 9.

The control unit 10 outputs a signal for designating the emission timing of the illumination light beams Lr, Lg, and Lb to the wavelength-swept light source 61. Furthermore, the control unit 10 outputs a signal for designating the vibration frequencies and the amplitudes, which are the specifications of the driving signals, to the signal generating section 71. The control unit 10 outputs the information regarding the emission timing of the illumination light beams Lr, Lg, and Lb and the information regarding the designation signal for the signal generating section 71, namely, information including the irradiation positions of the illumination light beams Lr, Lg, and Lb, to the image generating section 83.

The control unit 10 outputs the signal to the signal generating section 71 so that the signal generating section 71 generates waveform signals, as the two driving signals, which vibrate with phases different from each other by substantially 90° and whose amplitudes change in the form of sine waves, and so that the vibration periods of the two driving signals are proportional to the amplitudes.

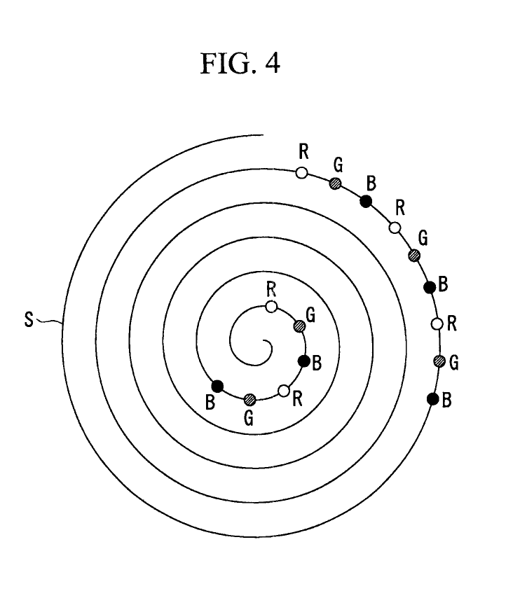

As shown in FIG. 3, two driving voltages in the X direction and the Y direction generated from these driving signals are alternating voltages whose amplitudes A are synchronous with each other and that change in the form of sine waves. As shown in FIG. 4, the actuator 4 receiving the two driving voltages scans the illumination light beams Lr, Lg, and Lb along a spiral scan trajectory S on the observation surface A.

In this case, the distal-end surface of the illumination fiber 2 is oscillated such that the oscillation period thereof, corresponding to a period T of each driving voltage, is proportional to the oscillation amplitude corresponding to the amplitude A of the driving voltage. Specifically, the frequency at which the illumination light beams Lr, Lg, and Lb are scanned is lower at the outer peripheral side of the spiral scan trajectory S so that the light beams are scanned at a fixed rate along the scan trajectory S. Thus, the three illumination light beams Lr, Lg, and Lb emitted from the wavelength-swept light source 61 at fixed time intervals are radiated at a fixed pitch along the scan trajectory S.

On the other hand, the control unit 10 displays the RGB image (color image) and the special-light observation image received from the image generating section 83 side-by-side on the monitor 9.

Next, the operation of the scanning endoscope device 1 having the above-described configuration will be described.

In order to observe the inside of a living organism by using the scanning endoscope device 1 according to this embodiment, the insertion section 5 is inserted into the living organism while the illumination light beams Lr, Lg, and Lb are emitted in that order from the wavelength-swept light source 61. The illumination light beams Lr, Lg, and Lb are scanned in a spiral pattern over the observation surface A within the living organism so as to illuminate the observation surface A, whereby an RGB image (color image) of the observation surface A and/or a special-light image is/are displayed on the monitor 9.

In this case, according to this embodiment, since the illumination light beams Lr, Lg, and Lb are radiated at a fixed pitch along the scan trajectory S, the illumination light beams are radiated with a uniform irradiation density along the entire scan trajectory. Thus, a peripheral area within the original image corresponding to the outer peripheral side of the scan trajectory S can be captured with the same resolution as that of a central area.

Furthermore, the use of a single light detector 81 for detecting the multiple return light beams Lr′, Lg′, and Lb′ is advantageous in terms of a simple configuration. Moreover, by using the single light detector 81 to temporally sample the signal intensities of the multiple return light beams Lr′, Lg′, and Lb′ in synchronization with the emission timing of the illumination light beams Lr, Lg, and Lb from the wavelength-swept light source 61, multiple two-dimensional images based on the respective illumination light beams Lr, Lg, and Lb can be generated. Thus, different colors are prevented from being displayed at misaligned positions (i.e., color misregistration) in the special-light image and the RGB image (color image) formed by superposing the two-dimensional images, thereby accurately reproducing the colors of the observation surface A.

As an alternative to this embodiment in which a color image and a narrow-band light image are observed, a color image and a fluorescence image may be observed.

For example, a material existing in the observation surface A is dyed or marked in advance by using a fluorochrome that can be excited by the blue illumination light beam Lb. When the blue illumination light beam Lb is radiated, fluorescence Lf from the fluorochrome is generated as a return light beam in addition to the blue return light beam Lb′. The excitation light is intermittently radiated onto the fluorochrome so that fading of the fluorochrome can be prevented.

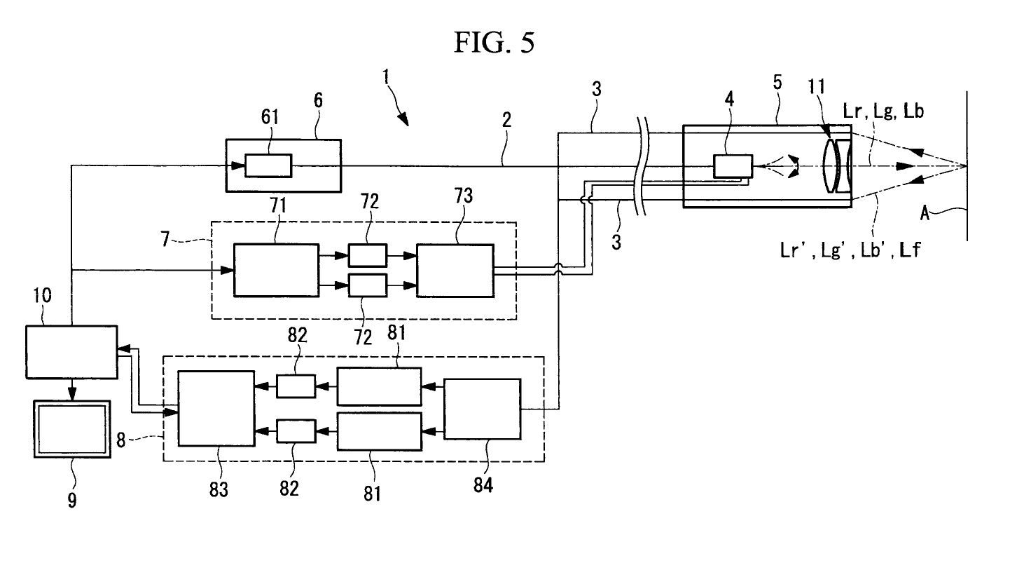

In this case, referring to FIG. 5, another light detector 81 for detecting the fluorescence Lf and a wavelength demultiplexer (wavelength branch section) 84 that is located at the front stage of the light detectors 81 and distributes the return light beams Lr′, Lg′, and Lb′ and the fluorescence Lf in accordance with the wavelengths are also provided. Consequently, the return light beam Lg′ and the fluorescence Lf that are simultaneously generated from the observation surface A are independently detected, so that the image generating section 83 can generate a B-image and a fluorescence image independently.

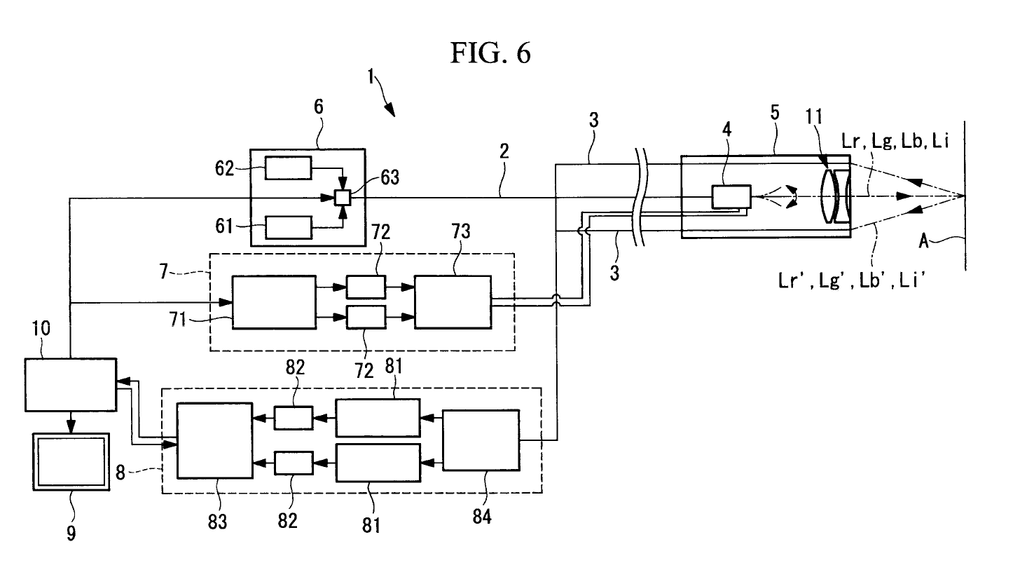

Furthermore, referring to FIG. 6, in this embodiment, another light source 62 may be provided in addition to the wavelength-swept light source 61, and the illumination light beams may be input to the illumination fiber 2 by switching between the wavelength-swept light source 61 and the additional light source 62 by using an optical-path switching section 63, such as a shutter. The additional light source 62 used may be, for example, a high-output near-infrared light source used in medical treatment.

Accordingly, since near-infrared light Li is radiated with a uniform density at any position on the observation surface A, the effect of treatment using the near-infrared light Li can be improved by accurately adjusting the irradiation amount of the near-infrared light Li.

In this case, the image generating section 83 may generate an IR image from return light Li′ of the near-infrared light Li. The control unit 10 may display the IR image and the RGB image (color image) side-by-side or in a superposed fashion on the monitor 9.

Furthermore, as described above, a target material in an area to be treated by using the near-infrared light Li may be dyed or marked in advance by using a fluorochrome that can be excited by any one of the illumination light beams Lr, Lg, and Lb. The control unit 10 may control the illumination unit 6 so as to make it radiate the near-infrared light Li only to an area corresponding to a fluorescence area within a generated fluorescence image.

Furthermore, although the wavelength-swept light source 61 is provided as a light source in this embodiment, a light source that releases steady light, such as a xenon lamp, and a wavelength changing section that changes the wavelength of light input to the illumination fiber 2 from the light source may alternatively be provided. The wavelength changing section may be formed of, for example, a filter turret having a band-pass filter that extracts light in a predetermined wavelength band from the light from the light source, a wavelength-tunable liquid-crystal filter, or an electro-optic crystal.

In this embodiment, the illumination unit 6 emits the illumination light beams Lr, Lg, and Lb with a fixed repetition period, and the control unit 10 controls the actuator 4 so that the reciprocating scan period is proportional to the scan amplitude of the illumination light beams Lr, Lg, and Lb. Alternatively, the actuator 4 may vibrate the illumination fiber 2 at a fixed frequency, and the control unit 10 may control the illumination unit 6 so that the repetition period is proportional to the scan amplitude of the illumination light beams Lr, Lg, and Lb.

Accordingly, since the illumination light beams Lr, Lg, and Lb are radiated at a fixed pitch along the scan trajectory S, the illumination light beams Lr, Lg, and Lb can be radiated with a uniform density onto the observation surface A.

Although a spiral scan method is described as an example of a method for scanning illumination light beams in this embodiment, the scan method is not limited to this method.

For example, if a method in the related art with a fixed reciprocating scan period is used in a Lissajous scan method or a propeller scan method in which the illumination light beams are scanned in a reciprocating manner in two axial directions while changing the amplitude, as in the spiral scan method, the distance between positions irradiated with the illumination light beams is increased in an area where the amplitude in the scan region is large, resulting in reduced resolution and notable color misregistration.

In contrast, with this embodiment, the reciprocating scan period is changed so that it is proportional to the scan amplitude of the illumination light beams, whereby the illumination light beams are radiated at a fixed pitch at any position along the scan trajectory. Then, the signals of return light beams are detected for the respective wavelengths in synchronization with the repetition period of the illumination light beams. Therefore, the irradiation density of the illumination light beams can be made uniform even when an image based on multiple illumination light beams is to be observed, thereby preventing the occurrence of reduced resolution and color misregistration in the area where the scan amplitude is large.

Furthermore, the configuration of the scanning endoscope device described in this embodiment is only an example; the configuration of the scanning endoscope device is not limited to that described above. For example, although the illumination light beams are two-dimensionally scanned by vibrating the distal end of the illumination fiber 2 in the two axial directions, the illumination light beams may alternatively be two-dimensionally scanned by oscillating a mirror (emission surface) in a reciprocating manner in the two axial directions.

### REFERENCE SIGNS LIST

1 scanning endoscope device
2 illumination fiber (light guiding section)
3 light-receiving fiber
4 actuator (driving section)
5 insertion section
6 illumination unit (light source unit)
7 driving unit
8 detecting unit
9 monitor
10 control unit (controller)
11 illumination optical system
31 light-receiving surface
61 wavelength-swept light source
71 signal generating section
72 D/A converting section
73 signal amplifying section
81 light detector (light detecting section)
82 A/D converting section
83 image generating section
84 wavelength demultiplexer (wavelength branch section)
A observation surface
Lr, Lg, Lb illumination light beams
Lr′, Lg′, Lb′ return light beams
S scan trajectory

1. A scanning endoscope device comprising:
an insertion section that is inserted into a subject;
a light source unit that repeatedly emits a plurality of illumination light beams in different wavelength bands in a certain order with a predetermined repetition period;
a light guiding section that is provided within the insertion section and has an emission surface that causes the illumination light beams from the light source unit to be emitted from an end of the insertion section;
a driving section that oscillates the emission surface in two axial directions, which intersect a longitudinal direction of the insertion section, in a reciprocating manner so as to two-dimensionally scan the illumination light beams;
a controller that controls at least one of the light source unit and the driving section so that an oscillation period of the emission surface and a oscillation amplitude of the emission surface are proportional to the predetermined repetition period of the illumination light beams, and that controls the driving section so that the oscillation period of the emission surface gradually changes and the oscillation period is proportional to the oscillation amplitude;
a light detecting section that detects return light beams from the subject; and
an image generating section that generates images of the return light beams in synchronization with the predetermined repetition period of the light source.

2. The scanning endoscope device according to claim 1,
wherein the light detecting section includes a plurality of light detection sections, and
wherein the scanning endoscope device further comprises a wavelength branch section that is provided at a front stage of the light detecting sections and that distributes the return light beams in accordance with wavelengths thereof.

3. The scanning endoscope device according to claim 1, wherein the light source unit includes a wavelength-swept light source that emits the illumination light beams while changing wavelengths thereof.
